(19) United States
(12) Patent Application Publication
(10) Pub. No.: US 2023/0309856 A1
(43) Pub. Date: Oct. 5, 2023

(54) METHOD FOR IDENTIFYING AND MONITORING ILLNESSES FROM GAS SAMPLES CAPTURED BY A DEVICE AND METHOD FOR TRAINING A NEURAL NETWORK TO IDENTIFY ILLNESSES FROM GAS SAMPLES CAPTURED BY A DEVICE

(71) Applicant: Nathalia MORAES DO NASCIMENTO, Rio de Janeiro (BR)

(72) Inventors: Nathalia MORAES DO NASCIMENTO, Feira de Santana (BR); Júllia MORAES DO NASCIMENTO, Feira de Santana (BR); Rheyller DE SOUZA VARGAS, São Vicente (BR)

(21) Appl. No.: 17/904,028

(22) PCT Filed: Feb. 10, 2021
(86) PCT No.: PCT/BR2021/050064

§ 371 Date: Jan. 4, 2023

(30) Foreign Application Priority Data
Feb. 11, 2020 (BR) 102020002883-9

(51) Int. Cl. A61B 5/08; G16H 50/20

(52) U.S. Cl. CPC A61B 5/082; G16H 50/20

(57) ABSTRACT

The invention relates to a method for identifying and monitoring diseases from gas samples captured by a device, comprising the steps of capturing at least one sample of gases present in the environment at a time prior to a gas sample capture from a user's blow, capturing the gas sample from a user's blow; capturing at least one sample of gases present in the environment at a time subsequent to the moment of capturing the blow; generating a data array including data related to the captured gas samples; using the data array as input of at least one neural network trained to associate at least one disease with a gas signature, configuring the neural network to indicate whether the data array used as input generates a positive or negative output for the at least one disease.

### FIELD OF THE INVENTION

The present invention relates to a method for identifying and monitoring diseases that allows the identification and automatic generation of gaseous signatures of diseases/microbiomes from the use of neural networks and machine learning algorithms.

### STATE OF THE ART

Various equipment, devices, and respective data processing methods for identifying diseases from gases exhaled by blowing are known in the state of the art, thus facilitating diagnostic processes in general.

In this sense, document WO2009/020647 stands out, disclosing a portable device for analyzing exhaled gases that measures various analytes from breathing or even blood glucose levels and transmits the data to a remote location, before or after analyzing them. Said device may comprise a storage medium, an analysis medium and a communication medium.

Document US20180338023, in turn, discloses a mobile device that may have an exhaled gas analysis module to detect properties related to different health conditions or even information regarding alcohol intake. Although the purpose of the application is aimed at a device for use with a mobile terminal, the equipment, when configured, has a gas and ambient sensor or sensors, a sensor reading unit, a processing unit, a user interface, a storage unit, and a communication unit.

US2006/0058697 shows a portable health status checking device comprising a breathing sensor, a processing unit, a memory, a user interface unit, and a communication unit. This document describes a device based on the detection of hydrogen, using tungsten oxide produced by chemical deposition process of metalorganic vapor. The device further comprises user voice identifiers.

Furthermore, the document US2008045825 describes a device for collecting exhaled gas in breath, comprising a control system for determining the concentration of glucose from gases and sensors that can adsorb the gases for quantitative analyses. The equipment also comprises a display, as well as a memory and a communication system.

Document CN108281201, in turn, describes a cloud-based microprocessor-based remote diagnostics system comprising an acquisition device, a user equipment, a cloud server and a terminal equipment, wherein the acquisition device with the cloud microprocessor is used for the acquisition of concentration data of organic matter in exhaled gas by a user and for wirelessly transmitting the acquired concentration data from the organic matter to the user equipment. The user equipment comprises a computer for uploading the received concentration data of the organic matter to the cloud server, and the cloud server is used to calculate and process the uploaded concentration data of the organic matter.

Also, as a representative of the state of the art, the Brazilian document BR 112013032313-2 can be cited, which describes an end-stream gas monitoring apparatus for monitoring gas in exhaled breath for diagnostic purposes, comprising a hydrogen sulfite gas sensor, carbon monoxide gas sensor, carbon dioxide gas sensor, hydrogen gas sensor, nitric oxide gas sensor, or nitrogen dioxide gas sensor, for example. The device also comprises a computer operationally coupled to the gas sensor component; a memory component operationally coupled to the computer; a database stored within the memory component and a means for transmitting said data.

### OBJECTIVES OF THE INVENTION

It is one of the objectives of the present invention to provide a method for identifying and monitoring diseases from gas samples captured by a device that allows the identification and automatic generation of gas signatures of diseases/microbiomes from the use of neural networks and machine learning algorithms.

It is another objective of the present invention to provide a method for identifying and monitoring diseases from gas samples captured by a device, which makes use of neural networks trained to represent the gas signatures of diseases, in order to analyze the concentration of gases identified for such diseases.

It is another objective of the present invention to provide a method for identifying and monitoring diseases from gas samples captured by a device that captures data at times before and after the user's blow, in order to identify any interference in the environment caused by the blow.

It is another objective of the present invention to provide a method for training a neural network to identify diseases from gas samples captured by a device.

### BRIEF DESCRIPTION OF THE INVENTION

The present invention achieves these and other objectives through a method for identifying and monitoring diseases from gas samples captured by a device, comprising:
a) capturing at least one sample of gases present in the environment at a time prior to a sample capture of gases from a blow from a user;
b) capturing the gas sample from a user's blow at a time later than the time of capture of step a);
C) capturing at least one sample of gases present in the environment at a time later than the time of capture of step b);
generating a data array including data related to the captured gas samples; and
using the data array as input to at least one neural network trained to associate at least one disease with a gas signature, the neural network being configured to indicate whether the data array used as input generates a positive or negative output, or an output corresponding to a discrete value between 0 and 1 for the at least one disease.

Steps a) and c) may further comprise capturing, along with the gas samples present in the environment, temperature, humidity, and air flow data; and step d) may comprise generating a data array including data related to the gas samples captured and the temperature, humidity, pressure, GPS, sound, altitude and air flow data captured in steps a) and c).

In one embodiment of the method of the invention, step a) comprises capturing a plurality of gas samples and a plurality of temperature, humidity and air flow data at different times prior to capturing the gas sample from a user's blow; and step c) comprises capturing a plurality of gas samples and a plurality of temperature, humidity, pressure, GPS, sound, altitude and air flow data at different times after capturing the gas sample from a user's blow.

In another embodiment, step d) further comprises converting the data array into a graphic image and step e) comprises using the converted graphic image from the data array as input to the at least one neural network.

The data array can be used in a plurality of neural networks, each neural network being trained to associate a different disease with a gas signature, each neural network being configured to indicate whether the data array used as input generates a positive or negative output, or an output with a discrete value between 0 and 1 for the different disease for which that neural network was trained. Thus, the method may comprise a step f) of generating a result report with the positive or negative outputs corresponding to each different disease.

In another embodiment, the data array can be used in a multiclass neural network, the multiclass neural network being trained to associate a plurality of diseases with a corresponding plurality of gas signatures and being configured to indicate whether the data array used as input generates a positive or negative output for each disease of the plurality of diseases. Therefore, the method may further comprise a step f) of generating a result report with the positive or negative outputs, or even between 0 and 1 for each disease of the plurality of diseases.

The output values, when between 0 and 1, can be converted to a positive/negative value for the identification of a disease among the plurality of diseases. Furthermore, discrete output values between 0 and 1 can be related to the level of a disease, and to factors such as glucose levels or the effect of a drug.

The present invention also contemplates a method of training a neural network for identifying and monitoring diseases from gas samples captured by a device, comprising:
providing a data set that includes, for each patient of a plurality of patients:
data related to at least one sample of gases present in the environment at a time prior to a sample collection of gases from a patient's blow;
data related to a gas sample from a patient's blow;
data related to at least one sample of gases present in the environment at a time later than the moment of capturing the gas sample from a patient's blow;
providing a second data set comprising data from different diagnosed diseases for each patient of the plurality of patients; and
automatically build, using an automatic machine learning algorithm, a neural network model that relates a diagnosed disease from the plurality of diagnosed diseases to a different gas signature.

In one embodiment, the method can build a plurality of neural network models that relate the plurality of diagnosed diseases to different gas signatures.

In another embodiment, the method can build a multiclass neural network that relates the plurality of diagnosed diseases to different gas signatures.

### BRIEF DESCRIPTION OF THE DRAWINGS

The present invention will be described in more detail below, with reference to the accompanying drawings, in which:

FIG. 1—schematically illustrates a gas capture device that uses a disease identification method according to an embodiment of the present invention;

FIG. 2—is a flowchart that schematically illustrates the data capture phase of the disease identification method according to an embodiment of the present invention;

FIG. 3—is a schematic illustration of the data array of the disease identification method according to an embodiment of the present invention;

FIG. 4—is a schematic illustration of the training phase of the disease identification method according to an embodiment of the present invention;

FIG. 5—is a schematic illustration of a neural network used by the disease identification method according to an embodiment of the present invention;

FIG. 6—is a schematic illustration of the storage of trained neural networks used by the disease identification method according to an embodiment of the present invention;

FIG. 7—is an illustration of a storage database of trained neural networks used by the method for identifying diseases according to an embodiment of the present invention;

FIG. 8—is an illustration of a multiclass neural network storage database used by the disease identification method according to an embodiment of the present invention;

FIG. 9—is a schematic illustration of the training phase of the disease identification method according to an embodiment of the present invention, using a multiclass neural network;

FIG. 10—is a schematic illustration of a multiclass neural network used by the method for identifying diseases according to one embodiment.

FIG. 11—is a schematic illustration of the analysis phase of the disease identification method according to an embodiment of the present invention,

FIG. 12—is a schematic illustration of the analysis phase of the disease identification method according to an embodiment of the present invention, wherein the method uses a plurality of neural networks;

FIG. 13—is a schematic illustration of the analysis phase of the disease identification method according to an embodiment of the present invention, wherein the method uses a multiclass neural network;

FIG. 14—is a tabulation of the data array of the disease identification method according to an embodiment of the present invention;

FIG. 15—is a plot of the data array of the disease identification method according to an embodiment of the present invention;

FIG. 16—is a schematic illustration of the disease identification method according to an embodiment of the present invention; and

FIG. 17—is a schematic illustration of the analysis phase of the disease identification method according to an embodiment of the present invention.

### DETAILED DESCRIPTION OF THE INVENTION

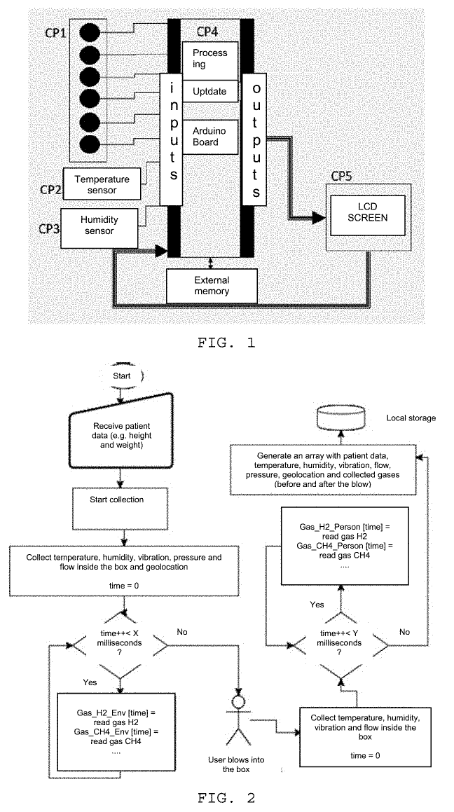

The present invention will be described below based on the embodiments of the invention illustrated in FIGS. 1 to 17.

In one embodiment, the present invention relates to a method for identifying and monitoring diseases from gas samples captured by a device.

The device of the present invention can be any suitable gas capture device. As illustrated in FIG. 1, in one embodiment, the device comprises a collection unit that receives the reading from the N gas sensors, humidity, temperature, GPS, sound, pressure, altitude and flow sensors (e.g. high-sensitivity portable anemometer, accelerometer or vibration sensor), a motherboard (Arduino, Intel, Galileo, Raspberry Pi); and a processing unit, which includes a set of trained artificial neural networks (ANN).

The sensors and gas (CP1) may include: H2, NH3, CO2, CH4, H2NO3, H2S, NO sensors, among others.

The method of the present invention comprises a data collection phase, responsible for the detection of gases in the environment and sample composition (input vector). The proposed method evaluates temperature, humidity, pressure, GPS, sound, altitude and flow and the gases before and after the blow from the patient/user, that is, data from the current environment and the environment after suffering interference from the blow are collected. Such pre-blow and post-blow analysis enables the present invention to dispense with the need for a controlled environment for uptake.

FIG. 2 illustrates the gas collection process in one embodiment of the method of the present invention. Thus, when starting the device, before starting the collection, the user can manually enter some patient information, such as weight, height and if he/she is a smoker. When starting the collection, the device will present a message asking the user to wait X milliseconds before blowing. During this time of X milliseconds, the system will collect the data of the current environment before it suffers interference by the user's blow. The data collected can be temperature, humidity, vibration, air flow inside the device, and a discrete reading of the gas sensors (reading of the sensors at time 1, time 2, and so on). After these X milliseconds are over, the system will prompt the user to blow on the external nozzle, and then start a new Y millisecond data collection.

in this way, the method can capture at least one sample of gas present in the environment at a time before a sample of gas from a user's blow and at least one sample of gases present in the environment at a time after the moment of capturing the user's blow. During the stages of capturing ambient gases in the moments before and after capturing the blow, data on temperature, humidity, pressure, GPS, sound, altitude, and air flow can be captured, along with the samples of gases present in the environment.

It should be understood that the method can capture any number of a plurality of samples at different times within X milliseconds before the blow and within Y milliseconds after the blow.

After capturing the data, the method generates a data array including data related to the captured gas samples and temperature and pressure data that, according to Clapeyron's equation (P.V=n.R.T), are important to know the number of moles of a particular gas analyte. In addition, temperature also influences the sensitivity of the sensor by increasing its conductivity or decreasing it depending on the material used in each sensor. It should be noted that this variation is predicted by OHM's law in the study of the resistivity of a metallic material and its conductivity. Humidity can also be measured, as it influences the sensitivity of the gas sensors.

The present invention can also make use of a GPS, which can map the altitude at which the user is. The altitude influences the pressure that, according to the equation of state of the ideal gas, must be measured to know the number of moles of a gaseous analyte.

The sound can be measured through a sensor, being used to know the state of the user's airways, such as, for example, if there are obstructions or not, to measure the intensity and frequency, since the mucous membranes released (sputum) during certain health conditions emit sound during blowing and breathing. During the blowing from the user, the air flow can also be measured, since it is able to tell the volume of analytes per unit of time and it is also an apparatus that contains a good part of the air inside a semi-closed container.

Thus, it is expected that the fact that a user's blow with more or less force increases the pressure inside the container, having a direct relationship with the identification of the number of moles of a given gas analyte.

Thus, the device used to implement the method of the present invention also uses a pressure measuring sensor to increase the reliability of other pressure measurements (direct or indirect) via data corroboration.

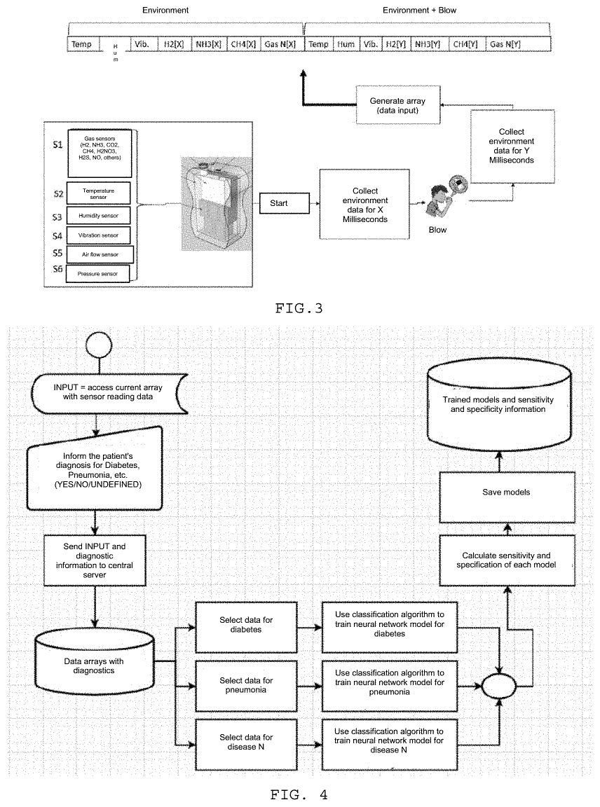

FIG. 3 exemplifies the data array that can be generated during the collection phase at the end of each execution of the device. The array contains data from both the moment before the blow and the moment after the blow.

The data array generated in the collection phase can be used both in the training phase (automatic generation of gas signatures) and in the device use phase (device with embedded neural network).

Thus, in the method of the present invention, the device uses the data array as input to at least one neural network trained to associate at least one disease with a gas signature, the neural network being configured to indicate whether the data array used as input generates a positive or negative output for the at least one disease.

In one embodiment of the present invention, the method comprises a plurality of neural networks, wherein each neural network is trained to associate a different disease with a gas signature. Thus, each neural network is configured to indicate whether the data array used as input generates a positive or negative output for the different disease for which that neural network was trained.

In one embodiment of the present invention, the method comprises a plurality of neural networks, wherein each neural network is trained to associate a different disease with a gas signature. Thus, each neural network is configured to indicate whether the data array used as input generates an output with a discrete value between 0 and 1 for the different disease for which that neural network was trained. The discrete values of the output can be converted to positive or negative in the identification of a disease.

In addition, the discrete output values between 0 and 1 can be used to relate factors, such as: patient's disease level, glucose level and effect of a drug.

In one embodiment of the invention, during neural network training, instead of just correlating the input data with a positive or negative value for a disease, one can also consider the levels of intensity of the disease, quantifying its stage, such as initial and advanced.

Likewise, training can also be performed considering the glucose level. Thus, the reference value to be used for training the network(s) will be a discrete value, rather than only positive and negative for a particular disease. Thus, the output of the neural network, which generates a value between 0 and 1, will be converted to the different glucose measurements, for example, the output of the neural network 0.5 can be converted to 120 mg/DL. In the case of monitoring the effect of a drug in order to create the database used for training the neural network, it is also possible, for a certain period of time, to store information regarding the time of drug administration by the patient and the stage of the disease. Thus, for the analysis of the drug effect, it is possible to convert the discrete value provided by the neural network, which is calculated from the input data, such as gases, temperature, and disease stage, into an estimate of time for drug administration.

In yet another embodiment, the method comprises a multiclass neural network, trained to associate a plurality of diseases with a corresponding plurality of gas signatures. Thus, the multiclass neural network is configured to indicate whether the data array used as input generates a positive or negative output for each disease of the plurality of diseases.

FIGS. 4 to 10 illustrate the training of artificial neural networks used in embodiments of the method of the present invention.

Thus, for the training of the networks, the device will use a first set of data that includes, for each patient of a plurality of patients, data related to at least one sample of gases present in the environment at a time prior to a sample collection of gases from a patient's blow, data related to a sample of gases from a patient's blow, and data related to at least one sample of gases present in the environment at a time subsequent to the moment of collection of the sample of gases from a patient's blow; and a second set of data that a second set of data comprising data of different diseases diagnosed for each patient of the plurality of patients (for example, diabetes, pneumonia, tuberculosis, etc.). This information will be stored in a common database, located on a central server.

After building the database, the method builds, using an automatic machine learning algorithm, at least one neural network model that relates a diagnosed disease of the plurality of diagnosed diseases to a different gas signature.

FIG. 4 illustrates the flowchart of this automatic gas signature generation process. The saved data, which contains a diagnosis for pneumonia, for example, will be used by an automatic machine learning (AUTO-ML) algorithm or also known as neural architecture search (ELSKEN, 2018), to automatically build a neural network for pneumonia analysis, until it reaches a desired sensitivity and specificity. This process is repeated for each disease. At the end of the automatic construction of the models, the model of each disease will be saved in a database, along with its sensitivity and specificity information. Once the connections and weights of the neural networks already trained have been identified, they can be converted into functions, in such a way that they are transferred to different devices.

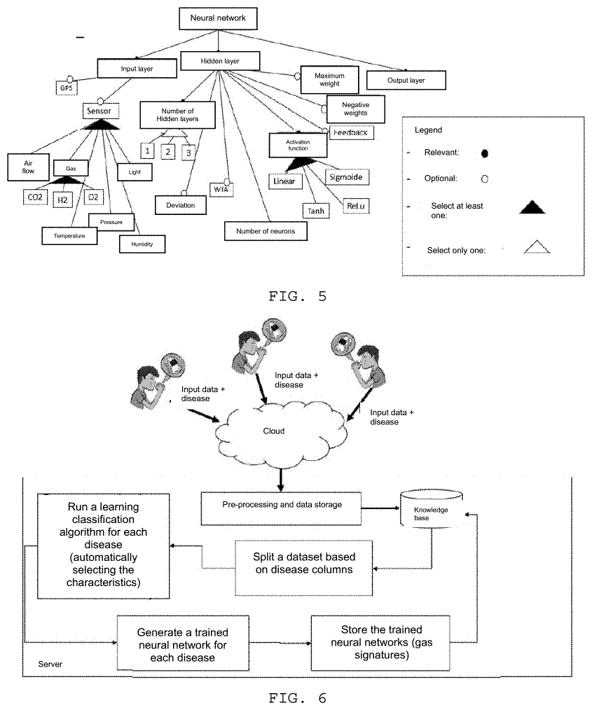

The process of automatic machine learning or neural architecture search, already known from the state of the art, consists of testing various neural network models automatically until finding a model that presents suitable results for a specific database. The models may vary according to the number of features (input variables), neural network architecture, activation function, etc. FIG. 5 shows the feature-oriented domain analysis (FODA) diagram, which is commonly used to represent configurable systems, to illustrate the characteristics that can vary in the generated neural networks. For example, the neural networks generated for the different diseases may vary in terms of the input sensors selected, the number of layers and the activation function. If a neural network does not consider the input of a particular sensor, the value obtained by the sensor will be ignored during the analysis performed with that neural network. It may happen that the neural network for diabetes considers only the minimum and maximum values read by the acetone sensor, while the neural network for tuberculosis considers all the discrete values that were obtained by all the gas sensors.

FIGS. 6, 7 and 8 present in more detail the system for automatic generation of gaseous signatures, which are represented by neural network models.

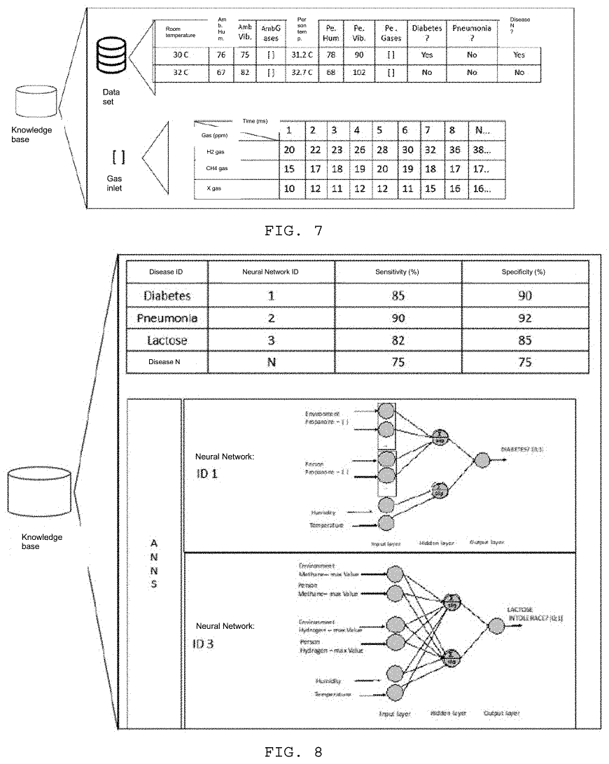

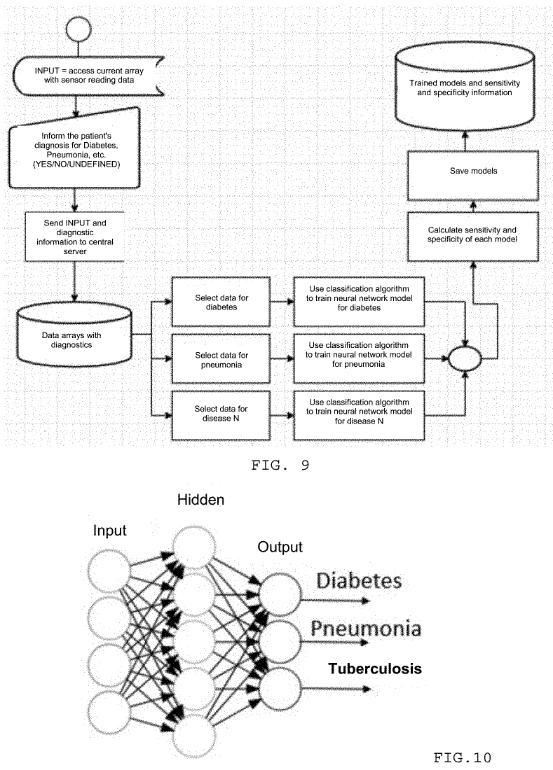

As illustrated in FIG. 6, all data collected during the training phase of the device is stored in a cloud repository. FIG. 7 provides details of how the data is stored in the database. Each row of the data table represents an array obtained in the collection phase, containing the information of the samples obtained before and after the user's blow. The data obtained from the gas sensors are stored discretely according to the time and reading interval established during the collection phase. FIG. 8 illustrates the neural network models that can be automatically generated for each of the diseases, according to the stored data. In the example presented, the neural network used to analyze diabetes is a neural network with three layers, two neurons hidden in the middle layer and sigmoid function. As input, this neural network considers only the readings obtained from the propane gas sensor (from the environment before and after the blow), from the temperature and humidity sensor. The neural network to analyze lactose intolerance, on the other hand, does not use the discrete reading of the gas sensors, but only the maximum reading of the gas sensors before and after the user blows into the device.

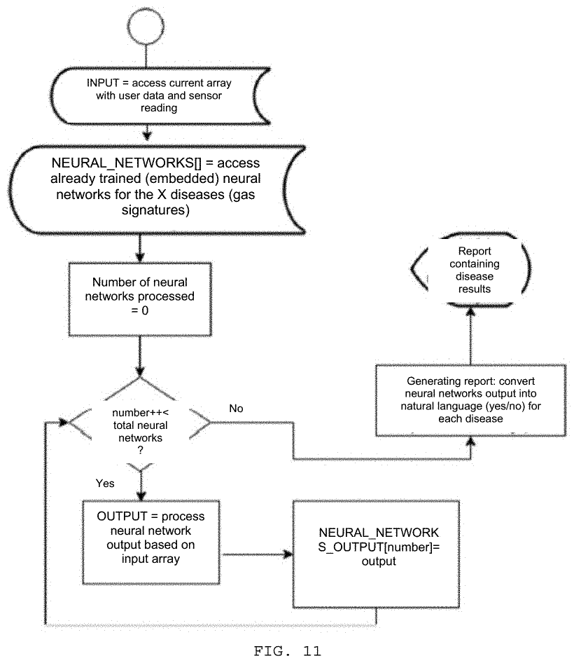

FIGS. 9 and 10 show the embodiment of the method that uses only a single multiclass neural network. As can be seen in these figures, the automatic signature generation system follows the same logic as that of the embodiment of the method that uses the plurality of neural networks.

After the training phase of the device, the neural network models stored in the database can be transferred to the device, so that they are embedded, and work offline and independently, without needing any type of connection.

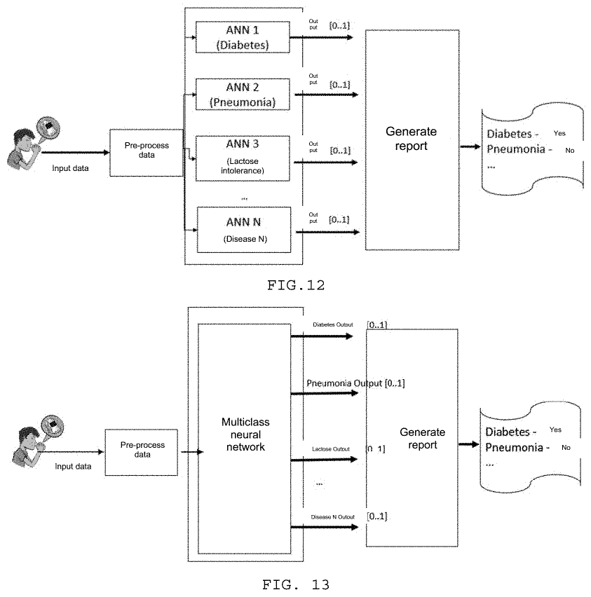

FIGS. 11 and 12 show the steps of the method with trained networks, in which the data are collected, according to the collection phase described above, and used as input to the N neural networks stored in the internal memory of the device. If the data array contains data A and B, but the neural network only considers the input A, the data B is automatically ignored by that neural network. After calculating the output of all stored neural networks using this array of data as input, the method will process the outputs and convert to natural language. For example, if the output of the diabetes neural network can take a value between zero and one, and the output is 0.95, then the output for diabetes is converted to “Yes”. After processing all outputs, the method generates a report to the user, containing the analysis of all diseases for which the networks were trained.

If the neural network(s) are trained with discrete values, such as the stage of a certain disease (e.g. initial and advanced), the output of the neural network can be converted to a discrete value, instead of just “Yes” and “No”. For example, tuberculosis is a disease that can have a clinical picture divided into three stages, the first being the initial stage, and the third being the most advanced stage of the disease. In this case, depending on the neural network training data, if the neural network output value for tuberculosis is a value between the interval [0; 0.1], this value can be converted to “No”, indicating the non-presence of the disease. If the output value of the neural network is between the range [0.11; 0.4], it can be converted to a textual information, indicating the presence of the first stage of the disease. The value between the range [0.41; 0.8] may indicate the second stage, as well as the value between [0.81; 1.0], which may indicate the most advanced stage. Likewise, if the system is trained to identify glucose, it can be trained to output both the “normal”, “pre-diabetic” and “diabetic” values as well as the person's own glycemic index value. In this case, given a data array containing the information from the gas, temperature and humidity sensors, the neural network will output a value between 0 and 1, and this value may indicate a glycemic index value corresponding to a value between the minimum and maximum glycemic indexes that were identified during the neural network training process.

FIG. 13 shows the steps of implementing the method using a multiclass neural network. As can be seen in this figure, for the multiclass neural network, more than one output neuron from the output bed may be active since a patient may have a positive output for more than one disease.

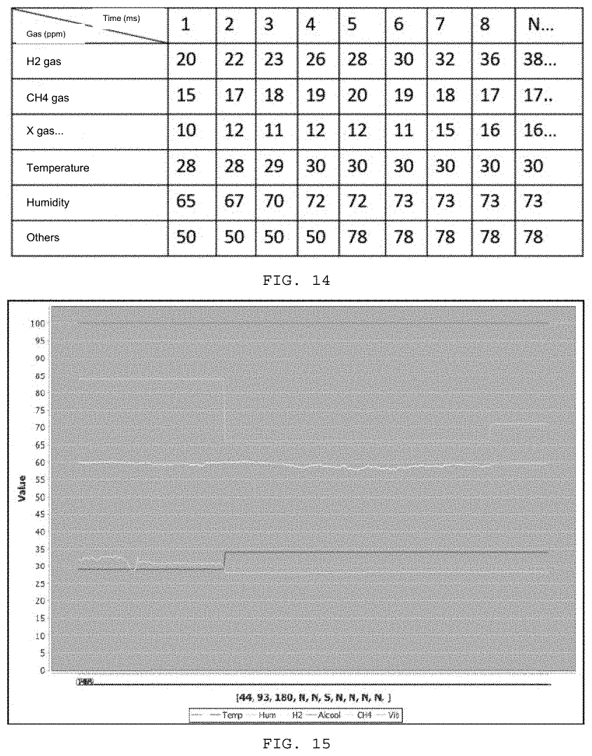

FIGS. 14 to 17 show an embodiment of the method of the present invention, in which the data array is converted into a graphic image, and it is the graphic image converted from the data array that is used as input to neural networks.

Thus, as represented in FIG. 17, the method has similar steps to the other embodiments already described, but it comprises generating graphic images from the data collected from all sensors and training a multiclass neural network for image processing.

FIG. 14 illustrates the data array containing the sensor reading at a time N. This array will be converted into a graphical image containing this data plotted at time N, as illustrated in FIG. 15. Before plotting, all data will be normalized to the same range of values.

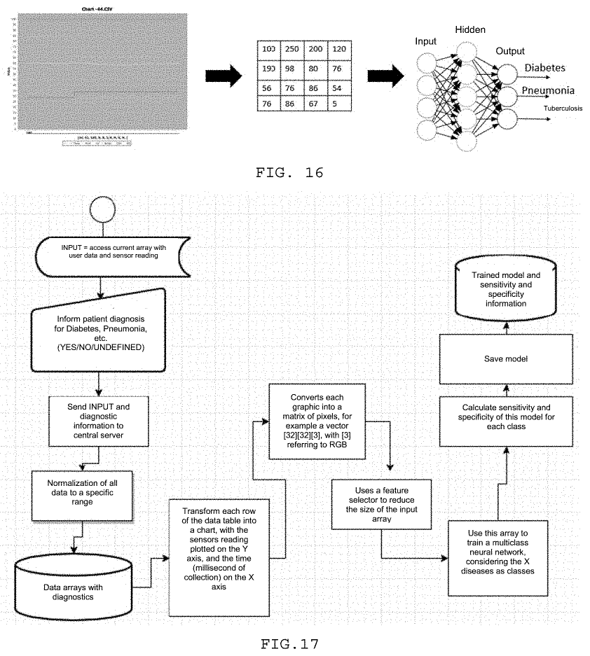

This graph will then be converted to a matrix of pixels, as illustrated in FIG. 16, which can be a matrix of 32 rows and 32 columns (32×32), for example, where the assumed values can be RGB (3 values between 0 and 255), or a simplified and proper representation of only one value between 0 and 255, since the image does not contain many colors, and the image can have a pre-treatment before being converted into a matrix of pixels. In addition, some parts of the graphic image are unnecessary for analysis, and therefore it is possible to apply a feature selection algorithm to reduce the size of this matrix. The advantage of using an image containing all the plotted data is that the number of neural network inputs can be reduced, depending on the number of sensors. For example, if we use a 32×32 input matrix, in which the colors are represented in a simplified way in only one value between 0 and 255, the number of neural network inputs will be 1024. In the current model, if the system has 10 sensors, and the collection performed 100 readings before blowing and 150 after blowing, the number of neural network inputs can be 2.500 ((100+150)*10), considerably increasing the training time.

Having described examples of embodiments of the present invention, it should be understood that the scope of the present invention encompasses other possible variations of the inventive concept described, being limited only by the content of the appended claims, including possible equivalents.

1. Method for identifying and monitoring diseases from gas samples captured by a device, comprising the following steps:
a) capturing at least one sample of gases present in an environment at a time prior to a sample capture of gases from a user's blow;
b) capturing the gas sample from a user's blow at a time later than the time of the capture in step (a);
c) capturing at least one sample of gases present in the environment at a time later than the time of capture of step (b);
d) generating a data array including data related to the captured gas samples;
e) using the data array as input to at least one neural network trained to associate at least one disease with a gas signature, the neural network being configured to indicate whether the data array used as input generates a positive or negative output for the at least one disease.

2. Method according to claim 1, wherein:
steps (a) and (c) further comprise capturing, together with samples of gases present in the environment, temperature, humidity and air flow data; and
step d) comprises generating a data array including data related to the gas samples captured and the temperature, humidity, pressure, GPS, sound, altitude, and air flow data captured in steps a) and c).

3. Method according to claim 2, wherein:
step a) comprises capturing a plurality of gas samples and a plurality of temperature, humidity, pressure, GPS, sound, altitude, and air flow data at different times prior to capturing the gas sample from a user's blow; and
step c) comprises capturing a plurality of gas samples and a plurality of temperature, pressure, GPS, altitude, sound and humidity and air flow data at different times after capturing the gas sample from a user's blow.

4. Method according to claim 3, wherein:
step d) further comprises converting the data array into a graphic image and step e) comprises using the converted graphic image from the data array as input to the at least one neural network.

5. Method according to claim 1, wherein step e) comprises using the data array in a plurality of neural networks, each neural network being trained to associate a different disease with a gaseous signature, each neural network being configured to indicate whether the data array used as input generates a positive or negative output for the different disease for which that neural network was trained.

6. Method according to claim 5, further comprising a step f) of generating a result report with the positive or negative outputs corresponding to at least one of: each different disease, the level of intensity of a disease, the blood glucose level or the treatment response to a particular drug.

7. Method according to claim 1, wherein step e) comprises using the data array in a multiclass neural network, the multiclass neural network being trained to associate a plurality of diseases with a corresponding plurality of gas signatures, and the neural network being configured to indicate whether the data array used as input generates a positive or negative output for each disease of the plurality of diseases.

8. Method according to claim 7, wherein it further comprises a step f) of generating a result report with the positive or negative outputs for each disease of the plurality of diseases.

9. Method for identifying and monitoring diseases from gas samples captured by a device, comprising the following steps:
f) capturing at least one sample of gases present in the environment at a time prior to a sample capture of gases from a user's blow;
g) capturing the gas sample from a user's blow at a time later than the time of the capture in step (a);
h) capturing at least one sample of gases present in the environment at a time later than the time of capture of step (b);
i) generating a data array including data related to the captured gas samples;
j) using the data array as input to at least one neural network trained to associate at least one disease with a gas signature, the neural network being configured to indicate whether the data array used as input generates a discrete value output between 0 and 1 for the at least one disease.

10. Method according to claim 9, wherein:
steps a) and c) also comprise capturing, together with the samples of gases present in the environment, temperature, humidity, pressure, GPS, sound, altitude, and air flow data; and
step d) comprises generating a data array including data related to the gas samples captured and the temperature, humidity, pressure, GPS, sound, altitude, and air flow data captured in steps a) and c).

11. Method according to claim 10, wherein:
step a) comprises capturing a plurality of gas samples and a plurality of temperature, humidity, pressure, GPS, sound, altitude, and air flow data at different times prior to capturing the gas sample from a user's blow; and
step c) comprises capturing a plurality of gas samples and a plurality of temperature, humidity, pressure, GPS, sound, altitude, and air flow data at different times after capturing the gas sample from the blow of a user.

12. Method according to claim 11, wherein:
step d) further comprises converting the data array into a graphic image and step e) comprises using the converted graphic image from the data array as input to the at least one neural network.

13. Method according to claim 9, wherein step e) comprises using the data array in a plurality of neural networks, each neural network being trained to associate a different disease with a gaseous signature, each neural network being configured to indicate whether the data array used as input generates a positive or negative output for the different disease for which that neural network was trained.

14. Method according to claim 13, wherein it further comprises a step f) of generating a result report with the discrete outputs between 0 and 1 corresponding to each different disease, or with the level of intensity of a disease, with the level of glucose in the blood or with the treatment response to a certain drug.

15. Method according to claim 9, wherein step e) comprises using the data array in a multiclass neural network, the multiclass neural network being trained to associate a plurality of diseases with a corresponding plurality of gaseous signatures, and the neural network being configured to indicate whether the data array used as input generates a positive or negative output for each disease of the plurality of diseases.

16. Method according to claim 15, wherein it further comprises a step f) of generating a result report with the discrete outputs between 0 and 1 for each disease of the plurality of diseases.

17. Method according to claim 9, wherein the discrete value of the outputs between 0 and 1 is related to the level of the disease, glucose levels or effect of a drug.

18. Method according to claim 9, wherein the discrete value of the outputs between 0 and 1 can be converted into positive and negative for disease identification of the plurality of diseases.

19. Method of training a neural network to identify diseases from gas samples captured by a device, comprising the following steps:
a) providing a data set that includes, for each patient of a plurality of patients:
data relating to at least one sample of gases present in the environment at a time prior to a sample collection of gases from a patient's blow;
data related to a gas sample from a patient's blow;
data relating to at least one sample of gases present in the environment at a time subsequent to the time of collection of the gas sample from a patient blow;
b) providing a second data set comprising data of different diagnosed diseases for each patient of the plurality of patients;
c) automatically building, using an automatic machine learning algorithm, a neural network model that relates one diagnosed disease of the plurality of diagnosed diseases to a different gas signature.

20. Method according to claim 9, wherein step c) comprises automatically constructing, using automatic machine learning algorithms, a plurality of neural network models that relate the plurality of diagnosed diseases to different gas signatures.

21. Method according to claim 9, wherein step c) comprises automatically constructing, using an automatic machine learning algorithm, a multiclass neural network that relates the plurality of diagnosed diseases to different gas signatures.
